(19) United States
(12) Patent Application Publication
(10) Pub. No.: US 2025/0025400 A1
(43) Pub. Date: Jan. 23, 2025

(54) COSMETIC COMPOSITION FOR CARING FOR THE SKIN

(71) Applicant: L'OREAL, Paris (FR)

(72) Inventors: Xiao CHEN, Shanghai (CN); Qing YANG, Shanghai (CN)

(73) Assignee: L'OREAL, Paris (FR)

(21) Appl. No.: 18/711,436

(22) PCT Filed: Dec. 29, 2021
(86) PCT No.: PCT/CN2021/142492

§ 371 Date: May 17, 2024

(51) Int. Cl. A61K 8/49; A61K 8/365; A61Q 5/00

(52) U.S. Cl. CPC A61K 8/494; A61K 8/365; A61Q 5/006; A61K 2800/48; A61K 2800/51

(57) ABSTRACT

It relates to a cosmetic composition for caring for the skin, comprising: (i) at least one N-substituted aminosulfonic acid compound; (ii) at least one hydroxy acid; and (iii) at least one penetration enhancer It also relates to a non-therapeutic method for caring for the skin, comprising applying the composition to the skin for a period of time and then rinsing the skin.

### TECHNICAL FIELD

The present invention relates to a cosmetic composition. In particular, the present invention relates to a cosmetic composition for caring for the skin, in particular the scalp. The present invention also relates to a non-therapeutic process for caring for the skin, in particular the scalp.

### BACKGROUND ART

The appearance of dandruff is troublesome both in terms of personal appearance and discomfort (itching, rash, etc.). Therefore, many individuals suffering from dandruff seek an effective and definitive treatment method. Anti-fungal agents are commonly used to control dandruff by eliminating or reducing the multiplication of resident yeast on the scalp. Common anti-fungal agents include climbazole, zinc pyrithione, and piroctone olamine, which can be included in an anti-dandruff composition.

For example, IN201927042237A discloses an antidandruff hair care composition comprising (i) 0.01 to 3% by weight zinc pyrithione; (ii) 1 to 5% by weight amino acid; and (iii) 0.1 to 5% by weight additional zinc compound.

IN202147030786A discloses a hair care composition comprising from 0.5 to 45% by weight of a salt of acyl glutamate and an anti-dandruff agent of piroctone olamine, wherein the salt of acyl glutamate and the anti-dandruff agent are present in a weight ratio of from 5:1 to 50:1, and wherein the composition does not comprise other anionic surfactants in addition to the salt of acyl glutamate.

However, after long time use of these anti-fungal agents, consumers feel that their scalp tends to be dry and fragile.

Consumers find treating dandruff with a shampoo to be convenient because such treatment fit into the consumers' regular routine. Anti-dandruff shampoos provide cleansing benefits to the hair while simultaneously treating dandruff.

However, many commercial available anti-dandruff shampoos are not effective enough as far as anti-dandruff performance is concerned.

Thus, there is a need to formulate a cosmetic composition for caring for the skin, in particular the scalp, which can relieve dandruff issue and consequences due to dandruff such as itchiness, erythema, etc., without disturbing scalp barrier function.

### SUMMARY OF THE INVENTION

An object of the present invention is to provide a composition for caring for the skin, in particular the scalp, which can relieve dandruff issue and consequences due to dandruff such as itchiness, erythema, etc., without disturbing scalp barrier function.

Another object of the present invention is to provide a non-therapeutic method for caring for the skin, in particular the scalp, which can relieve dandruff issue and consequences due to dandruff such as itchiness, erythema, etc, without disturbing scalp barrier function.

Accordingly, in a first aspect, the present invention provides a cosmetic composition for caring for the skin, comprising:
(i) at least one N-substituted aminosulfonic acid compound;
(ii) at least one hydroxy acid; and
(iii) at least one penetration enhancer.

In a second aspect, the present invention relates to a non-therapeutic method for caring for the skin, comprising applying the composition according to the first aspect of the present invention to the skin for a period of time and then rinsing the skin.

It was found that the composition according to the present invention can relieve dandruff issue and consequences due to dandruff such as itchiness, erythema, etc, without disturbing scalp barrier function.

It was also found that the composition according to the present invention is milde and stable with time at room temperature (25° C.).

Other subjects and characteristics, aspects and advantages of the present invention will emerge even more clearly on reading the detailed description and the examples that follow.

### BRIEF DESCRIPTION OF THE DRAWINGS

Implementations of the present invention will now be described, by way of example only, with reference to the attached figures, wherein:

FIG. 1 shows photos of the scalp of a subject with severe dandruff, wherein A: before the treatment; B: after being treated for 4 weeks.

FIG. 2 shows photos of the scalp of a subject with moderate dandruff, wherein A: before the treatment; B: after being treated for 4 weeks.

FIG. 3 shows the standard for scoring the dandruff on a scalp.

FIG. 4 shows the scalp dryness scale using a dermascore.

FIG. 5 shows the HEPES signal in stratum corneum upon application of composition of invention example 1, wherein A: after 1 minute; B: after 15 minutes.

FIG. 6 shows photos of the composition of invention example 1 after stored for 2 months, wherein A: stored at room temperature (25° C.), B: stored at 45° C.

FIG. 7 shows photos of the composition of invention example 4 after stored for 2 months, wherein A: stored at 45° C., B: stored at room temperature (25° C.).

### DETAILED DESCRIPTION OF THE INVENTION

In that which follows and unless otherwise indicated, the limits of a range of values are included within this range, in particular in the expressions “between . . . and . . . ” and “ranging from . . . to . . . ”.

Moreover, the expression “at least one” used in the present description is equivalent to the expression “one or more”.

Throughout the instant application, the term “comprising” is to be interpreted as encompassing all specifically mentioned features as well as optional, additional, unspecified ones. As used herein, the use of the term “comprising” also discloses the embodiment wherein no features other than the specifically mentioned features are present (i.e. “consisting of”).

Unless otherwise specified, all numerical values expressing amount of ingredients and the like which are used in the description and claims are to be understood as being modified by the term “about”. Accordingly, unless indicated to the contrary, the numerical values and parameters described herein are approximate values, which are capable of being changed according to the desired purpose as required.

Unless otherwise defined, all technical and scientific terms used herein have the same meaning as commonly understood by those skilled in the art the present invention belongs to. When the definition of a term in the present description conflicts with the meaning as commonly understood by those skilled in the art the present invention belongs to, the definition described herein shall apply.

For the purposes of the present invention, the term “skin” is intended to cover facial skin, body skin, mucous membranes such as the lips, and the scalp. The scalp is most particularly considered according to the present invention.

All percentages in the present invention refer to weight percentage, unless otherwise specified.

According to the first aspect, the present invention provides a cosmetic composition for caring for the skin, in particular the scalp, comprising:
(i) at least one N-substituted aminosulfonic acid compound;
(ii) at least one hydroxy acid; and
(iii) at least one penetration enhancer.

### N-Substituted Aminosulfonic Acid Compounds

According to the first aspect, the composition according to the present invention comprises at least one N-substituted aminosulfonic acid compound.

Suitable N-substituted aminosulfonic acid compounds include, but are not limited to, N,N-bis[2-hydroxyethyl]-2-aminoethanesulfonic acid, N-2-hydroxyethylpiperazine-N′-2-ethanesulfonic acid, 3-[N-morpholino]propanesulfonic acid, piperazine-N,N′-bis[2-ethanesulfonic]acid, 3-[N-tris(hydroxymethyl)methylamino]-2-hydroxypropanesulfonic acid, 2-[N-morpholino]ethanesulfonic acid, N-(2-acetamido)-2-aminoethanesulfonic acid, and N-tris(hydroxymethyl)methyl-2-aminoethanesulfonic acid. A mixture of these acids may also be used.

Preferably, the N-substituted aminosulfonic acid compound is selected from N-2-hydroxyethylpiperazine-N′-2-ethanesulfonic acid, piperazine-N,N′-bis[2-ethanesulfonic]acid, and a combination thereof.

More preferably, the N-substituted aminosulfonic acid compound is N-2-hydroxyethylpiperazine-N′-2-ethanesulfonic acid, also called hydroxylethylpiperzine ethanesulfonic acid.

The inventors have found that using N-substituted aminosulfonic acid compounds, in particular, hydroxylethylpiperzine ethanesulfonic acid can ameliorate dandruff condition without disturbing scalp barrier function.

As a commercial product of N-substituted aminosulfonic acid compounds, mention can be made of hydroxylethylpiperzine ethane sulfonic acid sold under the name HEPES-LUV by the company TAIWAN HOPAX.

Advantageously, the N-substituted aminosulfonic acid compound is present in the composition in an amount ranging from 0.1 wt. % to 15 wt. %, preferably from 1 wt. % to 10 wt. %, more preferably from 3 wt. % to 7.5 wt. %, most preferably from 5 wt. % to 7.5 wt. %, relative to the total weight of the composition.

### Hydroxy Acids

According to the first aspect, the composition according to the present invention comprises at least one hydroxy acid.

Hydroxy acid suitable for the composition according to the present invention can be selected from alpha-hydroxy acids and beta-hydroxy acids.

The term “alpha-hydroxy acid” is understood to mean a carboxylic acid having at least one hydroxyl functional group occupying an alpha-position on said acid (carbon adjacent to a carboxylic acid functional group).

Suitable alpha hydroxy acids includes glycolic acid, citric acid, lactic acid, methyllactic acid, glucuronic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid, mandelic acid, phenyllactic acid, gluconic acid, galacturonic acid, aleuritic acid, ribonic acid, tartronic acid, tartaric acid, malic acid, fumaric acid.

Preferably, the alpha hydroxy acid is selected from lactic acid, glycolic acid, tartaric acid, mandelic acid, citric acid, and a combination thereof.

More preferably, the alpha hydroxy acid in the composition includes glycolic acid.

As a commercial product of alpha hydroxy acid, mention can be made of glycolic acid sold under the name KELTROL CG-T by the company CP KELCO.

The term “beta-hydroxy acid” is understood to mean a carboxylic acid having a hydroxyl functional group and a carboxylic functional group separated by two carbon atoms.

Suitable beta hydroxy acids include salicylic acid, propionic acid, beta-hydroybutyric acid, beta-hydroxy beata-methylbutyric acid, carnitine, derivatives thereof, and combinations thereof.

Preferably, the beta hydroxy acid is selected from salicylic acid and its derivative of the formula:

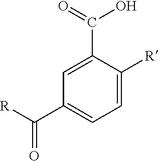

wherein R is a linear, branched or cyclic saturated aliphatic group or an aliphatic unsaturated group containing one or a number of double bonds, which may or may not be conjugated, these groups containing from 2 to 22, preferably 3 to 11 carbon atoms and being able to be substituted for example by at least one substituent selected from (a) halogen atoms, (b) the trifluoromethyl group, (c) hydroxyl groups in the free form or esterified by an acid having from 1 to 6 carbon atoms or (d) a carboxyl functional group which is free or esterified by a lower alcohol having from 1 to 6 carbon atoms;

R′ is a hydroxyl group or an ester functional group of the following formula:

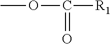

wherein R₁is a linear or branched saturated or unsaturated aliphatic group having from 1 to 18 carbon atoms.

Preferred salicylic acid derivatives include 5-n-octanoyl salicylic acid (capryloyl salicylic acid), 5-n-decanoyl salicylic acid, 5-n-dodecanoyl salicylic acid, 5-n-heptyloxy salicylic acid and 4-n-heptyloxy salicylic acid.

More preferably, the beta hydroxy acid is selected from salicylic acid, 5-n-octanoyl salicylic acid, 5-n-decanoyl salicylic acid, 5-n-dodecanoyl salicylic acid, 5-n-heptyloxy salicylic acid, 4-n-heptyloxy salicylic acid, and combination thereof. Most preferably, the beta hydroxy acid is salicylic acid.

For the purpose of the present invention, it is preferred that the hydroxy acid used in the composition comprises at least one alpha hydroxy acid.

Advantageously, the hydroxy acid is present in an amount ranging from 0.1 wt. % to 10 wt. %, preferably from 0.1 wt. % to 6 wt. %, and more preferably from 1 wt. % to 6 wt. %, relative to the total weight of the composition.

### Penetration Enhancers

According to the first aspect, the composition according to the present invention comprises at least one penetration enhancer.

The penetration enhancer suitable for use in the composition according to the present invention includes, but are not limited to
alcohols, alkanols, alkanones such as benzyl alcohol, decanol, ethanol, octanol, and propanol, oleyl alcohol;
polyols and esters thereof such as 1,2,6 hexanetriol, polyethylene glycol, propylene glycol monocaprylate, propylene glycol monolaurate;
fatty acids such as lauric acid, oleic acid, and valeric acid;
fatty acid esters such as ethyl oleate, isopropyl myristate, diisopropyl adipate, and methylpropionate;
amides and other nitrogenous compounds such as diethanolamine, dimethylacetamide, dimethylformamide, ethanolamine, 1-methyl-2-pyrrolidone, 2-pyrrolidone, triethanolamine, and urea;
ethers such as diethylene glycol monoethyl ether, polyethylene glycol octadecyl ether, polyoxyethylene lauryl ether and diethylene glycol monomethyl ether;
pyrrolidones such as 2-pyrrolidone;
1-substituted azacycloheptan-2-ones, such as 1-n-dodecylcyclazacycloheptan-2-one;
sulfoxides such as decylmethylsulfoxide and dimethylsulfoxide, and
a combination thereof.

Prefearbly, the penetration enhancer is selected from diethylene glycol monoethyl ether, oleyl alcohol, propylene glycol, diisopropyl adipate, ethanol, benzyl alcohol, isopropyl myristate, triethanolamine, polyethylene glycol octadecyl ether, polyoxyethylene lauryl ether, and a combination thereof.

More preferably, the penetration enhancer is ethanol.

Advantageously, the penetration enhancer is present in an amount ranging from 1 wt. % to 50 wt. %, preferably from 10 wt. % to 30 wt. %, and more preferably from 10 wt. % to 20 wt. %, relative to the total weight of the composition.

### Hydrophilic Thickeners

Preferably, the composition according to the present invention comprises at least one hydrophilic thickener.

It was found that with the presence of a hydrophilic thickener, a higher viscosity is obtained, which is beneficial for stability and application of the composition.

The viscosity of the composition according to the invention can be measured at 25 C, using a ProRheo R180 viscometer equipped with a spindle M1 or M2 rotating at 200 rpm. Preferably, the composition of the present invention has a viscosity of from 15 UD (Deviation Units) to 60 UD, preferably from 20 UD to 55 UD, measured at 25° C. using a Rheomat R180 viscometer equipped with a spindle M1 or M2 rotating at 200 rpm.

The term “hydrophilic thickener” is intended to mean a compound capable of increasing the viscosity of the aqueous phase of the composition.

The hydrophilic thickeners may be used alone or in combination.

As hydrophilic thickeners, mention may in particular be made of water-soluble or water-dispersible thickening polymers. They may in particular be chosen from:
polyvinylpyrrolidone,
polyvinyl alcohol,
modified or unmodified carboxyvinyl polymers, such as the products sold under the name Carbopol® (CTFA name: carbomer) by the company Goodrich;
homopolymers or copolymers of acrylic acid or methacrylic acid or salts thereof and esters thereof, and in particular the products sold under the names Versicol F® or Versicol K® or Salcare SC95 by the company Allied Colloid, Ultrahold 8® by the company Ciba-Geigy, polyacrylates and polymethacrylates, such as the products sold under the names Lubrajel and Norgel by the company Guardian or under the name Hispagel by the company Hispano Chimica, polyacrylic acids of Synthalen K type;
polyacrylamides;
copolymers of acrylic acid and of acrylamide sold in the form of their sodium salt under the names Reten® by the company Hercules, poly (sodium methacrylate) sold under the name Darvan N° 7® by the company Vanderbilt, the sodium salts of polyhydroxycarboxylic acids sold under the name Hydagen F® by the company Henkel;
homopolymers and copolymers of 2-acrylamido-2-methylpropanesulfonic acid, which are optionally crosslinked and/or neutralized, for instance the poly (2-acrylamido-2-methylpropanesulfonic acid) sold by the company Clariant under the name Hostacerin AMPS® (CTFA name: ammonium polyacryldimethyltauramide);
crosslinked anionic acrylamide/AMPS copolymers, in the form of a W/O emulsion, such as those sold under the name Sepigel 305 (CTFA name: Polyacrylamide/C13-14 Isoparaffin/Laureth-7) and under the name Simulgel 600 (CTFA name: Acrylamide/Sodium acryloyldimethyltaurate copolymer/Isohexadecane/Polysorbate 80) by the company SEPPIC;
polyacrylic acid/alkyl acrylate copolymers of Pemulen type;
polysaccharide biopolymers, for instance xanthan gum, guar gum, gum Arabic, locus bean gum, acacia gum, scleroglucans, chitin derivatives and chitosan derivatives, carrageenans, gellans, alginates, or celluloses such as microcrystalline cellulose, carboxymethylcellulose, hydroxymethylcellulose, hydroxyethylcellulose and hydroxypropylcellulose. Mentions maybe made of, for example, xanthan gum sold under the trade name Keltrol CG-T by the company CP Kelco; and hydroxyethylcellulose sold under the trade name NATROSOL 250 HHR by the company ASHLAND.
hydrophilic fumed silicas obtained by high-temperature hydrolysis of a volatile silicon compound in an oxyhydrogen flame, producing a finely divided silica. The hydrophilic silicas have a large number of silanol groups at their surface. Such hydrophilic silicas are, for example, sold under the names Aerosil 130®, Aerosil 200®, Aerosil 255®, Aerosil 300® and Aerosil 380® by the company Degussa, or Cab-O-Sil HS-5®, Cab-O-Sil EH-5®, Cab-O-Sil LM-130®, Cab-O-Sil MS-55® and Cab-O-Sil M-5® by the company Cabot. They preferably have a particle size that can be nanometric to micrometric, for example ranging from about 5 to 200 nm;
hydrophilic clays;
associative polymers, for instance the PEG-150/stearyl alcohol/SMDI copolymer sold under the name Aculyn 46 by Rohm & Haas, or the steareth-100/PEG-136/HDI copolymer sold under the name Rheolate FX 1100 by Elementis;
and mixtures thereof.

Preferably, the hydrophilic thickener is selected from polysaccharide biopolymers.

More preferably, the hydrophilic thickener is selected from xanthan gum, guar gum, gum Arabic, locus bean gum, acacia gum, scleroglucans, carrageenans, gellans, alginates, microcrystalline cellulose, carboxymethylcellulose, hydroxymethylcellulose, hydroxyethylcellulose, hydroxypropylcellulose, and a combination thereof.

Most preferably, the hydrophilic thickener comprises a combination of xanthan gum and hydroxyethylcellulose.

Preferably, the hydrophilic thickener is present in the composition in an amount of from 0.01 wt. % to 2 wt. %, preferably from 0.1 wt. % to 1 wt. %, more preferably from 0.1 wt. % to 0.8 wt. %, relative to the total weight of the composition.

### Chelating Agent

Preferably, the composition according to the present invention comprises at least one chelating agent.

More preferably the chelating agent is selected from aminocarboxylic acids and salts thereof.

The salts are especially alkali metal, alkaline-earth metal, ammonium and substituted ammonium salts.

The chelating agents may be selected in particular from the compounds having the following INCI name:
diethylenetriaminepentaacetic acid (DTPA),
ethylenediaminedisuccinic acid (EDDS) and trisodium ethylenediamine disuccinate such as Octaquest E30 from INNOSPEC ACTIVE CHEMICALS,
ethylenediaminetetraacetic acid (EDTA),
ethylenediamine-N, N′-diglutaric acid (EDDG),
glycinamide-N,N′-disuccinic acid (GADS),
2-hydroxypropylenediamine-N,N′-disuccinic acid (HPDDS),
ethylenediamine-N,N′-bis (ortho-hydroxyphenylacetic acid) (EDDHA),
N,N′-bis (2-hydroxybenzyl) ethylenediamine-N,N′-diacetic acid (HBED),
nitrilotriacetic acid (NTA),
methylglycine diacetic acid (MGDA),
N-2-hydroxyethyl-N,N-diacetic acid and glyceryl imino diacetic acid (as described in documents EP-A-317 542 and EP-A-399 133),
iminodiacetic acid-N-2-hydroxypropyl sulfonic acid and aspartic acid N-carboxymethyl N-2-hydroxypropyl-3-sulfonic acid (as described in EP-A-516 102),
beta-alanine-N,N′-diacetic acid, aspartic acid-N,N′-diacetic acid and aspartic acid-N-monoacetic acid (described in EP-A-509 382),
chelating agents based on iminodisuccinic acid (IDSA) (as described in EP-A-509 382),
ethanoldiglycine acid, and
tetrasodium glutamate diacetate (GLDA) such as Dissolvine GL38, 45S or GL-47-S from Akzo Nobel.

Among the chelating agents mentioned, ethylenediamine-tetraacetic acid (EDTA), diethylenetriaminepentaacetic acid (DTPA), S, S′-ethylenediaminedisuccinic acid (EDDS), trisodium ethylenediamine disuccinate, ethylenediaminetetramethylenephosphonic acid (EDTMP), tetrasodium glutamate diacetate (GLDA), and a combination thereof, are preferably used.

Most preferably, the chelating agent is tetrasodium glutamate diacetate.

It was found that with the presence of a chelating agent, the stability and the appearance of the composition can be improved; the appearance of the composition remains unchanged after 1 month's storage.

Advantageously, the chelating agent is present in the composition in an amount ranging from 0.01 wt. % to 0.5 wt. %, preferably from 0.05 wt. % to 0.3 wt. %, and more preferably from 0.1 wt. % to 0.25 wt. %, relative to the total weight of the composition.

### Aqueous Phase

The composition according to the present invention comprises an aqueous phase.

Preferably, the aqueous phase is a continuous phase.

The aqueous phase of the present invention comprises water.

The aqueous phase may also comprise water-miscible organic solvents (at room temperature of 20-25° C.), for instance polyols such as C2-C6 polyols, more particularly caprylyl glycol, butylene glycol, propanediol, hexylene glycol, glycerin; glycol ethers (especially containing from 3 to 16 carbon atoms) such as mono-, di- or tripropylene glycol (C1-C4) alkyl ethers, mono-, di-or triethylene glycol (C1-C4) alkyl ethers, and mixtures thereof.

According to the invention, the aqueous phase is present in an amount ranging from 70 wt. % to 90 wt. %, preferably from 75 wt. % to 85 wt. %, relative to the total weight of the composition.

### Additional Adjuvants or Additives

The composition of the present invention may comprise conventional cosmetic adjuvants or additives, for instance fragrances, preserving agents (for example, potassium sorbate, chlorphenesin and phenoxyethanol), pH regulators (for example citric acid, sodium hydroxide, potassium hydroxide), and mixtures thereof.

The skilled in the art can select the amount of the additional adjuvants or additive so as not to adversely affect the final use of the composition according to the present invention.

According to a particularly preferred embodiment, the present invention provides a cosmetic composition for caring for the skin, comprising, relative to the total weight of the composition:
(i) from 3 wt. % to 7.5 wt. % of N-2-hydroxyethylpiperazine-N′-2-ethanesulfonic acid;
(ii) from 1 wt. % to 6 wt. % of glycolic acid;
(iii) from 10 wt. % to 20 wt. % of ethanol;
(iv) from 0.1 wt. % to 0.8 wt. % of at least one hydrophilic agent selected from polysaccharide biopolymers; and
(v) from 0.1 wt. % to 0.25 wt. % of at least one chelating agent selected from ethylenediamine-tetraacetic acid, diethylenetriaminepentaacetic acid, S,S′-ethylenediaminedisuccinic acid, trisodium ethylenediamine disuccinate, ethylenediaminetetramethylenephosphonic acid, tetrasodium glutamate diacetate, and a combination thereof.

### Galenic Form and Use

Preferably, the composition of the present invention is in the form of serum.

In some preferred embodiments, the composition according to the present invention has a higher viscosity.

According to a preferred embodiment, the composition of the present invention has a viscosity of from 15 UD (Deviation Units) to 60 UD, preferably from 20 UD to 55 UD, measured at 25° C. using a Rheomat R180 viscometer equipped with a spindle M1 or M2 rotating at 200 rpm.

According to the second aspect, the present invention provides a non-therapeutic method for caring for the skin comprising applying the composition according to the first aspect of the present invention to the skin for a period of time and then rinsing the skin.

In particular, the skin is the scalp.

The following examples serve to illustrate the present invention without, however, being limiting in nature.

### EXAMPLES

Main raw materials used, trade names and supplier thereof are listed in Table 1.

TABLE 1

| INCI Name | Trade Name | Supplier |
|---|---|---|
| HYDROXYETHYLPIPERAZINE | HEPES-LUV | TAIWAN HOPAX |
| ETHANE SULFONIC ACID | | |
| GLYCOLIC ACID | GLYPURE 70 | CHEMOURS |
| ETHANOL | ETHYL ALCOHOL 99N | AROMA HOLLY LIMITED |
| XANTHAN | KELTROL CG-T | CP KELCO |
| TETRASODIUM GLUTAMATE | DISSOLVINE GL-47-S | AKZO NOBEL (NOURYON) |
| DIACETATE | | |
| HYDROETHYL CELLULOSE | NATROSOL 250 HHR CS | ASHLAND |

### Invention Examples 1-5 and Comparative Example 1

Compositions according to invention examples (IE.) 1-5 and comparative example (CE.) 1 were prepared according to the contents given in Table 2 (the contents are expressed as weight percentages of active material relative to the total weight of each composition, unless otherwise indicated).

TABLE 2

| | IE. 1 | IE. 2 | IE. 3 | IE. 4 | IE. 5 | CE. 1 |
|---|---|---|---|---|---|---|
| Components | wt. % | wt. % | wt. % | wt. % | wt. % | wt. % |
| HYDROXYETHYLPIPERAZINE | 5 | 5 | 5 | 5 | 5 | 0 |
| ETHANE SULFONIC ACID | | | | | | |
| GLYCOLIC ACID | 1.4 | 1.4 | 1.4 | 1.4 | 1.4 | 0 |
| EHTANOL | 10 | 10 | 10 | 10 | 10 | 0 |
| XANTHAN | 0.12 | 0.5 | 0.375 | 0.12 | 0.12 | 0.12 |
| HYDROETHYL CELLULOSE | 0 | 0 | 0.375 | 0 | 0 | 0 |
| TETRASODIUM GLUTAMATE | 0.2 | 0.2 | 0.2 | 0 | 0.2 | 0.2 |
| DIACETATE | | | | | | |
| POTASSIUM SORBATE | 0.1 | 0.1 | 0.1 | 0.1 | 0.1 | 0.1 |
| CAPRYLYL GLYCOL | 0.3 | 0.3 | 0.3 | 0.3 | 0.3 | 0.3 |
| BUTYLENE GLYCOL | 6 | 6 | 6 | 6 | 6 | 6 |
| PROPANEDIOL | 2 | 2 | 2 | 2 | 2 | 2 |
| SODIUM HYDROXIDE | 0.5 | 0.5 | 0.5 | 0.5 | 0.25 | 0 |
| CITRIC ACID | 0 | 0 | 0 | 0 | 0 | 0.7 |
| WATER | QS100 | QS100 | QS100 | QS100 | QS100 | QS100 |
| pH | 4.6 | 4.6 | 4.6 | 4.6 | 4 | 5.3 |

### Preparation Process:

The above compositions were prepared as follows:
1). adding xanthan gum and hydroxyethyl cellulose (if any) into water little by little to avoid agglomeration and stirring well until an uniform mixture was obtained;
2). adding glycolic acid and hydroxyethylpiperazine ethane sulfonic acid (if any) into the mixture and stirring well;
3). adding ethanol (if any), caprylyl glycol, butylene glycol and propanediol little by little into the mixture;
4). adding potassium sorbate and tetrasodium glutamate diacetate into the mixture; and
5). measuring and adjusting pH with sodium hydroxide and citric acid (if any) to obtain a composition.

The compositions obtained are in the form of serum.

### Example 2: Evaluation of compositions

The anti-dandruff efficacy, mildness, cleansing performance, and scalp barrier function and formula mildness, active penetration, viscosity, stability, of compositions prepared in Example 1 were characterized.

### Anti-Danruff Efficacy

The anti-danruff efficacy of composition of invention example 1 was evaluated by a consumer test and a clinical study as follows.

### Consumer Test

Study design: 4 weeks treatment, 15 subjects of 18-40 years old with moderate to severe dandruff, they are commercial anti-dandruff shampoo users.

Firstly, the composition of invention example 1 was applied on dirty dry scalp for 15 minutes, then the hair was washed with a commercial anti-dandruff shampoo. Such a treatment was carried out 3-4 times/week.

FIG. 1 shows photos of the scalp of a subject with severe dandruff: (a) before the treatment; (b) after being treated for 4 weeks.

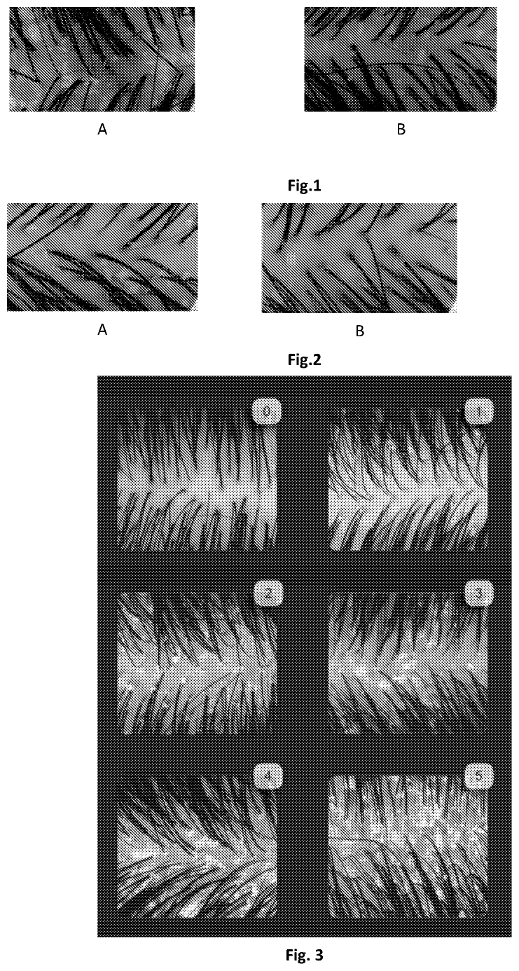

FIG. 2 shows photos of the scalp of a subject with moderate dandruff: (a) before the treatment; (b) after being treated for 4 weeks.

After 4 weeks' treatment, the subjects were invited to provide feedback on the dandruff issue laxation effect.

According to the subjects' feedback, as compared with only using the commercial anti-dandruff shampoo to wash the hair, after the subjects used the composition of invention example 1 and the commercial anti-dandruff shampoo for 2 weeks, the dandruff size was reduced, the dandruff quantity was decreased, dandruff syndrome including itchiness and erythema became better, the dandruff issue was under control after 2-3 weeks' application. Meanwhile, with application of the composition of invention example 1, the scalp was well moisturized and the greasy feeling on scalp can be pushed back for 0.5-1 day. The moisturized and itchiness-free scalp lasts well between 2 hair washes, which allows soothing and grease-free scalp for more than 1 day, there is almost no acne/bumpy scalp during product trial. The hair was well cleansed and had a volumized look.

### Clinical Study

Design: 8 subjects, the composition of invention example 1 (test) vs the composition of comparative example 1 (placebo), half head test, 7 days treatment.

The composition of invention example 1 and the composition of comparative example 1 were applied on dirty dry scalp of one half head and the other half head respectively for 15 minutes, then the hair was washed with a neutral shampoo. Such a treatment was carried out on every the other day once per day.

Test points: just before the first wash (TO), 24 h after first wash/just before the second wash (T24 h), 48 h after the first wash/just before the second wash, just before the third wash (T48 h), and just before the fifth wash (T8d).

The dandruff score within 0-5 was given at each test point based on the standard shown in FIG. 3.

The change of the score at T24 h, T48 h, and T8d over the score of TO was calculated and summarized in Table 3.

TABLE 3

| Score change vs T0 | T24 h | T48 h | T8 d |
|---|---|---|---|
| Half head-test (IE.1) | −0.61 | −0.59 | −0.98 |
| Half head-placebo (CE.1) | −0.55 | −0.53 | −0.97 |
| P-value | 0.821 | 0.451 | 0.007 (S) |

(S)-statistically significant at 95% CL

The results of the adhesive dandruff grade show that as compared with composition of comparative example 1, composition of invention example 1 can deliver anti-dandruff benefit during 7 days usage.

### Scalp Barrier Function and Cleansing Performance

The scalp barrier function and cleansing performance of the composition of invention example 1 was evaluated as follows.

### Study Design:

2 groups of subjects: test group and control group
Test group: 42 subjects of 20-40 years old with oily scalp;
Control group: 42 subjects of 20-40 years old with oily scalp.
During 6 week treatment, for the test group, the composition of invention example 1 was applied on dirty dry scalp for 15 minutes, then the hair was washed with a neutral shampoo; and for the control group, the hair was washed with the neutral shampoo without treating with the composition of invention example 1 beforehand. Such a treatment was carried out on once per day.

Transepidermal water loss (TEWL)

Transepidermal water loss (TEWL) was determined with a vapometer just before the first wash (T0), 24 h after the first wash/just before the second wash (T24 h), just before the 8^{th} wash (T2 wk), and just before the 15^{th} wash (T4 wk). Lower TEWL indicates better barrier function.

The results are summarized in Table 4.

TABLE 4

| TEWL Mean | T0 | T24 h | T2 wk | T4 wk |
|---|---|---|---|---|
| Test group | 26.8 | 25.8 | 25.3 | 23.4 ↓(S) |
| Control group | 22.9 | 22.6 | 24.1 | 28.1 ↑ |

↑: significant increase vs. T0 at 95% CL
↓: significant decrease vs T0 at 95% CL
S: significant different from control group at 95% CL

It can be seen that scalp barrier function was significantly improved at T4 wk for the test group; Control cell scalp barrier function was significantly decreased at T4 wk for the control group.

It can be also seen that the scalp barrier function was effectively improved at T4 wk with application of the composition of invention example 1.

### Scalp Sebum:

The sebum level was determined with a sebum meter just before the first wash (TO), 24 h after the first wash (T24 h), just before the 8^{th} wash (T2 wk), and just before the 15^{th} wash (T4 wk).

The results are summarized in Table 5.

TABLE 5

| Sebum Mean | T0 | T24 h | T2 wk | T4 wk |
|---|---|---|---|---|
| Test group | 84.9 | 80.6 | 91.3 | 91.8 |
| Control group | 64.0 | 62.1 | 73.5 | 82.7 ↑ |

↑: significant increase vs. T0 at 95% CL
↓: significant decrease vs T0 at 95% CL

It can be seen that scalp sebum maintained across time points for the test group, and scalp serum was significantly increased at T4 wk for the control group.

### Scalp Dryness:

The scalp dryness within 0-9 was determined with a dermascore just before the first wash (T0), 24 h after the first wash (T24 h), just before the 8^{th} wash (T2 wk), and just before the 15^{th} wash (T4 wk). Lower scalp dryness indicates better effect.

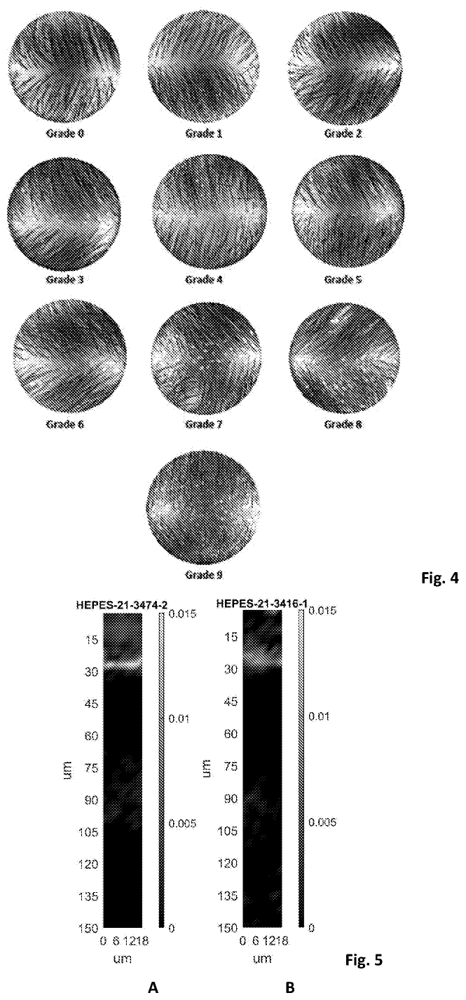

FIG. 4 shows the scalp dryness scale using a dermascore.

The results are summarized in Table 6.

TABLE 6

| Dryness Mean | T0 | T24 h | T2 wk | T4 wk |
|---|---|---|---|---|
| Test group | 2.2 | 1.2 ↓ | 1.5 ↓ | 1.5 ↓ |
| Control group | 1.7 | 1.0 ↓ | 1.7 | 1.4 |

↑: significant increase vs. T0 at 95% CL
↓: significant decrease vs T0 at 95% CL

It can be seen that scalp dryness was significantly improved across time point for the test group, scalp dryness was significantly improved at T24 h for the control group. As compared with the control group, a better cleansing performance was demonstrated for the test group using the composition of invention example 1.

In addition, for the test group, it was found that with the composition of invention example 1, a clean and whitened scalp can be observed, the dead cells softened by the serum can be easily rubbed off, there was a light, breathable, and cooling scalp feeling was obtained.

### Mildness

The mildness of compositions of invention example 1 and 5 were evaluated as follows:

The tested composition was injected into a Day-6 Epickin model from the company Shanghai EPISKIN Biotechnology Co., Ltd, the cell viability was determined after 18 hours incubation.

The cell viability was calculated as follows:
Cell viability (%)=N_{after}/N_{before}* 100%
N_{before}: the number of live cells before each treatment;
N_{after}: the number of live cells after each treatment.

The higher cell viability, the greater cellular survival is.

If the cell viability is greater than 50%, then the composition tested is proved to be mild enough for the scalp.

The results are summarized in Table 7.

TABLE 7

| Formula | mean viability % | SD |
|---|---|---|
| NgC (no treatment) | 100.0 | 0.0 |
| IE.1 | 64.8 | 8.6 |
| IE.5 | 66.9 | 4.3 |

The results show that the compositions of invention examples 1 and 5 are mild for the scalp.

### Penetration Ability

The penetration ability of the compositions of invention example 1 was evaluated with a Raman confocal laser scanning microscope as follows:
Material: 3 cm×3 cm porcine skin
Formula Application: 6 ul/0.8 cm×0.8 cm, topical 1 &15 minutes, 32° C.
Sample processing: cryo-section, 30 um thick/section

### Raman Confocal Parameters

Equipment: LabRam HR Evolution (Horiba Jobin-Yvon)
Raman Laser: 532nm
Objective: 50X
Step size: 3 um/point,
Scan area: 21 um (X axis) X 150 um (Y axis)
Pixel: 2 um/point

FIG. 5shows the HEPES (hydroxyethylpiperazine ethane sulfonic acid) signal in stratum corneum upon application of composition of invention example 1, wherein A: after 1 minute; B: after 15 minutes.

It can be seen from the comparison of the HEPES signal in stratum corneum after 1 minute of application and 15 minutes application, the composition of invention example 1 can be penetrated into the stratum corneum upon 15 minutes application.

### Viscosity

The viscosity was measured at 25° C., using a Rheomat R180 viscometer equipped with a M1 or M2 spindle, the measurement being performed after 10 minutes of rotation of the spindle in the composition (after which time stabilization of the viscosity and of the spin speed of the spindle are observed), at a shear rate of 200 rpm.

The viscosities were summarized in Table 8.

TABLE 8

| Properties | IE. 1 | IE. 2 | IE. 3 | IE. 4 | IE. 5 | CE. 1 |
|---|---|---|---|---|---|---|
| Viscosity | 53 | 21.5 | 32.7 | 53 | 52 | 49 |
| (UD) | (M1) | (M2) | (M2) | (M1) | (M1) | (M1) |

### Stability

The compositions of invention example 1 and 4 were stored at room temperature (25° C.).

FIG. 6 shows photos of the composition of invention example 1 after stored for 2 months, wherein A: stored at room temperature (25° C.), B: stored at 45° C.

It can be seen that the composition of invention example 1 did not show obvious change in appearance after stored at 25° C. and 45° C. for 2 months.

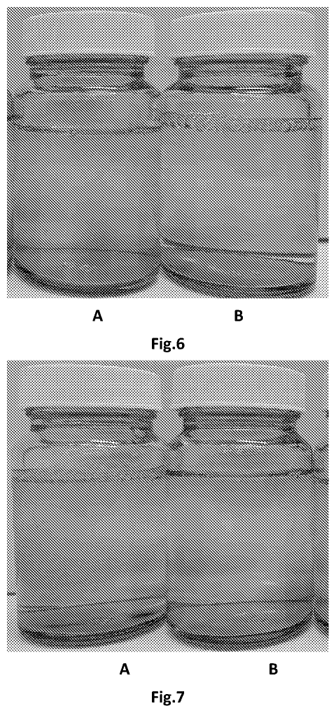

FIG. 7 shows photos of the composition of invention example 4 after stored for 2 months, wherein A: stored at 45° C., B: stored at room temperature (25° C.).

It can be seen that the composition of invention example 4 did not show obvious change in appearance after stored at 25° C. for 2 months, but turned yellow after stored at 45° C. for 2 months.

It can be seen that the composition of invention example 1 has a better stability than that of the composition of invention example 4.

1. A cosmetic composition for caring for the skin, comprising:
(i) at least one N-substituted aminosulfonic acid compound;
(ii) at least one hydroxy acid; and
(iii) at least one penetration enhancer.

2. The composition according to claim 1, wherein the N-substituted aminosulfonic acid compound is selected from N,N-bis[2-hydroxyethyl]-2-aminoethanesulfonic acid, N-2-hydroxyethylpiperazine-N′-2-ethanesulfonic acid, 3-[N-morpholino] propanesulfonic acid, piperazine-N,N′-bis[2-ethanesulfonic]acid, 3-[N-tris (hydroxymethyl) methylamino]-2-hydroxypropanesulfonic acid, 2-[N-morpholino] ethanesulfonic acid, N-(2-acetamido)-2-aminoethanesulfonic acid, N-tris (hydroxymethyl) methyl-2-aminoethanesulfonic acid, and a combination thereof.

3. The composition according to claim 1, wherein the N-substituted aminosulfonic acid compound is present in the composition in an amount ranging from 0.1 wt. % to 15 wt. %, relative to the total weight of the composition.

4. The composition according to claim 1, wherein the hydroxy acid is selected from alpha-hydroxy acids and beta-hydroxy acids.

5. The composition according to claim 1, wherein the hydroxy acid is present in an amount ranging from 0.1 wt. % to 10 wt. %, relative to the total weight of the composition.

6. The composition according to claim 1, wherein the penetration enhancer is selected from the group consisting of
benzyl alcohol, decanol, ethanol, octanol, and propanol, oleyl alcohol;
polyols and esters thereof;
fatty acids;
fatty acid esters;
amides and other nitrogenous compounds;
ethers;
pyrrolidones;
1-substituted azacycloheptan-2-ones;
sulfoxides, and
a combination thereof.

7. The composition according to claim 1, wherein the penetration enhancer is present in an amount ranging from 1 wt. % to 50 wt. %, relative to the total weight of the composition.

8. The composition according to claim 1, further comprising at least one hydrophilic thickener.

9. The composition according to claim 8, wherein the at least one hydrophilic thickener is selected from polysaccharide biopolymers, and a combination thereof.

10. The composition according to claim 8, wherein the at least one hydrophilic thickener is present in the composition in an amount of from 0.01 wt. % to 2 wt. %, relative to the total weight of the composition.

11. The composition according to claim 1, further comprising at least one chelating agent.

12. The composition according to claim 11, wherein the chelating agent is selected from aminocarboxylic acids and salts thereof.

13. The composition according to claim 11, wherein the at least one chelating agent is present in the composition in an amount ranging from 0.01 wt. % to 0.5 wt. %, relative to the total weight of the composition.

14. The composition according to claim 1 comprising, relative to the total weight of the composition:
(i) from 3 wt. % to 7.5 wt. % of N-2-hydroxyethylpiperazine-N′-2-ethanesulfonic acid;
(ii) from 1 wt. % to 6 wt. % of glycolic acid;
(iii) from 10 wt. % to 20 wt. % of ethanol;
(iv) from 0.1 wt. % to 0.8 wt. % of at least one hydrophilic agent selected from polysaccharide biopolymers; and
(v) from 0.1 wt. % to 0.25 wt. % of at least one chelating agent selected from ethylenediamine-tetraacetic acid, diethylenetriaminepentaacetic acid, S,S′-ethylenediaminedisuccinic acid, trisodium ethylenediamine disuccinate, ethylenediaminetetramethylenephosphonic acid, tetrasodium glutamate diacetate, and a combination thereof.

15. A non-therapeutic method for caring for the skin, comprising applying the composition according to claim 1 to the skin for a period of time and then rinsing the skin.

16. The composition according to claim 4, wherein the hydroxy acid is selected from the group consisting of glycolic acid, citric acid, lactic acid, methyllactic acid, glucuronic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid, mandelic acid, phenyllactic acid, gluconic acid, galacturonic acid, aleuritic acid, ribonic acid, tartronic acid, tartaric acid, malic acid, fumaric acid.

17. The composition according to claim 8, wherein the at least one hydrophilic thickener is selected from the group consisting of xanthan gum, guar gum, gum Arabic, locus bean gum, acacia gum, scleroglucans, carrageenans, gellans, alginates, microcrystalline cellulose, carboxymethylcellulose, hydroxymethylcellulose, hydroxyethylcellulose, hydroxypropylcellulose, and a combination thereof.

18. The composition according to claim 11, wherein the chelating agent is selected from the group consisting of diethylenetriaminepentaacetic acid, ethylenediaminedisuccinic acid, trisodium ethylenediamine disuccinate, ethylenediaminetetraacetic acid, ethylenediamine-N, N′-diglutaric acid, glycinamide-N,N′-disuccinic acid, 2-hydroxypropylenediamine-N,N′-disuccinic acid, ethylenediamine-N,N′-bis (ortho-hydroxyphenylacetic acid), N,N′-bis (2-hydroxybenzyl) ethylenediamine-N,N′-diacetic acid, nitrilotriacetic acid, methylglycine diacetic acid, N-2-hydroxyethyl-N,N-diacetic acid, glyceryl imino diacetic acid, iminodiacetic acid-N-2-hydroxypropyl sulfonic acid, aspartic acid N-carboxymethyl N-2-hydroxypropyl-3-sulfonic acid, beta-alanine-N,N′-diacetic acid, aspartic acid-N,N′-diacetic acid, aspartic acid-N-onoacetic acid, chelating agents based on iminodisuccinic acid, ethanoldiglycine acid, tetrasodium glutamate diacetate, and combinations thereof.
